(19) United States
(12) Patent Application Publication
(10) Pub. No.: US 2005/0123603 A1
(43) Pub. Date: Jun. 9, 2005

(54) Natural menaquinone 7 compositions

(75) Inventors: Jostein Dalland, Haslum (NO); Asgeir Saebo, Eiosnes (NO)

Correspondence Address: MEDLEN & CARROLL, LLP;Suite 350, 101 Howard Street, San Francisco, CA, 94105, US

(73) Assignee: Natural ASA, Lysaker (NO)

(21) Appl. No.: 10/950,783

(22) Filed: Sep. 27, 2004

Related U.S. Application Data
(60) Provisional application No. 60/506,117, filed on Sep. 26, 2003.
(60) Provisional application No. 60/512,587, filed on Oct. 17, 2003.
(60) Provisional application No. 60/554,040, filed on Mar. 16, 2004.

(51) Int. Cl. A61K 35/78; A61K 31/12; A61K 9/64

(52) U.S. Cl. 424/456; 424/776; 424/439; 514/682

(57) ABSTRACT

The present invention relates to the field of human nutrition, and in particular to pharmaceutical compositions and nutritional supplements comprising an effective daily dose of menaquinone 7. In particular, the present invention provides pharmaceutical compositions, nutritional supplements and food products that comprise from about 10 to about 200 micrograms of menaquinone 7. In some embodiments, the menaquinone 7 is provided in a natto extract.

The application claims the benefit of U.S. Prov. Appl. 60/506,117, filed Sep. 26, 2003, U.S. Prov. Appl. 60/512,587, filed Oct. 17, 2003, and U.S. Prov. Appl. 60/554,040, filed Mar. 16, 2004.

### FIELD OF THE INVENTION

The present invention relates to the field of human nutrition, and in particular to compositions and nutritional supplements comprising an effective daily dose of menaquinone 7.

### BACKGROUND OF THE INVENTION

Natto is a traditional Japanese food consisting of fermented soybeans. Natto is produced by growing Bacillus subtilis natto, a subspecies of Bacillus subtilis, on the surface of steamed soybean. During this process, the active ingredients of Natto (such as menaquinone-7) are synthesized and secreted by the bacteria.

Natto is known from the writings of Buddist monks more than 2000 years ago. Generations of Japanese have known of the curative effects of the dish. Scientific studies have been ongoing for more than 100 years and have documented the effect of Natto on osteoporosis, cardiovascular disease, and gastrointestinal disease. About 7.5 billion packages of Natto are eaten every year in Japan and consumption is increasing steadily. Japanese health authorities have expended huge resources on promoting regular use, including making Natto part of the school breakfast.

Scientists have identified menaquinone 7 as one of the active ingredients of Natto. Menaquinone 7 is a form of vitamin K2. Vitamin K2, which exists in several forms, is a fat-soluble vitamin found in fermented products such as cheese. Natto contains more K2, in particular menaquinone 7, than any other known source.

The typical dosage of Natto is approximately 80 grams per day. Due to the fact it is fermented, people of many cultures may object to the consumption of this much material. Extracts of Natto are known. However, to achieve the recommended dosage of these products, it is often necessary to consume several tablets, which is often inconvenient to the user.

Accordingly, what is needed in the art are pharmaceutical compositions that deliver an effective amount of the active ingredients of Natto in a single daily dose.

### SUMMARY OF THE INVENTION

The present invention relates to the field of human nutrition, and in particular to pharmaceutical compositions and nutritional supplements comprising an effective daily dose of menaquinone 7.

Accordingly, in some embodiments, the present invention provides an oral delivery vehicle comprising an effective daily dose of menaquinone 7, wherein the effective daily dose is from about 10 to about 200 micrograms of menaquinone 7. The present invention is not limited to menaquinone 7 derived from any particular source. In some embodiments, the menaquinone 7 is derived from natto. In further preferred embodiments, the oral delivery vehicle further comprises natto kinase. The present invention is not limited to any particular oral delivery vehicle. Indeed, a variety of oral delivery vehicles are contemplated, including, but not limited to, tablets and gel capsules. In further embodiments, the oral delivery vehicle comprises an enteric coating.

In other embodiments, the present invention provides a food product comprising added menaquinone 7. The present invention is not limited to food products containing any particular amount of menaquinone 7. In some embodiments, the food products comprise an effective daily dose of menaquinone 7, wherein the effective daily dose is from about 10 to about 200 micrograms of menaquinone 7. The present invention is not limited to menaquinone 7 derived from any particular source. In some embodiments, the menaquinone 7 added to the food product is derived from natto. In further preferred embodiments, the food product further comprises natto kinase.

In further embodiments, the present invention provides a nutritional supplement comprising an effective daily dose of menaquinone 7, wherein the effective daily dose is from about 10 to about 200 micrograms of menaquinone 7. The present invention is not limited to nutritional supplements containing menaquinone 7 derived from any particular source. In some embodiments, the menaquinone 7 in the nutritional supplement is derived from natto. In further preferred embodiments, the nutritional supplement further comprises natto kinase.

In still other embodiments, the present invention provides methods comprising: a) providing i) a subject, and ii) the oral delivery vehicle of Claim 1, and b) administering the oral delivery vehicle to the subject. The present invention is not limited to any particular mode of administration. In some embodiments, the mode of administration is oral. The present invention is not limited to menaquinone 7 derived from any particular source. In some embodiments, the menaquinone 7 is derived from natto. In further preferred embodiments, the oral delivery vehicle further comprises natto kinase. The present invention is not limited to any particular oral delivery vehicle. Indeed, a variety of oral delivery vehicles are contemplated, including, but not limited to, tablets and gel capsules. In further embodiments, the oral delivery vehicle comprises an enteric coating.

In still further embodiments, the present invention provides methods of relieving bone pain comprising: a) providing i) a patient, and ii) a composition comprising an effective amount of menaquinone 7, b) administering the composition to the patient under conditions such that the bone pain is reduced. The present invention is not limited to the administration of any particular amount of menaquinone 7. In some embodiments, the effective dose of menaquinone 7 is about 10 to about 200 micrograms. The present invention is not limited to any particular mode of administration. In some embodiments, the mode of administration is oral. The present invention is not limited to menaquinone 7 derived from any particular source. In some embodiments, the menaquinone 7 is derived from natto. In further preferred embodiments, the oral delivery vehicle further comprises natto kinase. The present invention is not limited to any particular oral delivery vehicle. Indeed, a variety of oral delivery vehicles are contemplated, including, but not limited to, tablets and gel capsules. In further embodiments, the oral delivery vehicle comprises an enteric coating.

In some embodiments, the present invention provides an oral delivery vehicle comprising an effective daily dose of menaquinone 7, wherein the effective daily dose supports a circulating concentration of menaquinone-7 in the body of about 0.1 to 50 ng menaquinone-7 per milliliter of serum.

In still further embodiments, the present invention provides methods of decreasing under-carboxylated osteocalcin in a subject comprising: a) providing i) a subject, and ii) a composition comprising an effective amount of menaquinone 7, b) administering said composition to said subject under conditions such that said under-carboxylated osteocalcin is reduced.

In still other embodiments, the present invention provides methods of decreasing bone turnover in a subject comprising: a) providing i) a subject, and ii) a composition comprising an effective amount of menaquinone 7, b) administering said composition to said subject under conditions such that said bone turnover is reduced.

In still further embodiments, the present invention provides methods of increasing serum menaquinone 7 levels to greater than 6000 pmol/L in a subject comprising: a) providing i) a subject, and ii) the oral delivery vehicle of Claim 1, b) administering said oral delivery vehicle to said subject under conditions such that serum menaquinone levels are increased to about 6000 pmol/L.

### DESCRIPTION OF THE FIGURES

FIG. 1: MK-7 in serum after natto consumption.

FIG. 2: MK-7 in serum after natto extract capsule consumption.

FIG. 3: MK-7 in individual subjects following natto or capsule consumption.

FIG. 4: A plot of the mean values for the groups.

FIG. 5A-5C: The mean serum vitamin K concentration (absolute values) in three groups: the K1-treated group (5A), the natto K2-treated group (5B) and the controls (5C).

FIG. 6: The effects of pharmacological K1 and natto MK-7 preparation on the total vitamin K status of the subjects.

FIG. 7: The treatment-induced changes in serum undercarboxylated osteocalcin (ucOC).

FIG. 8: The treatment-induced changes in serum carboxylated osteocalcin.

FIG. 9: The treatment-induced changes in the vitamin K status of bone, expressed as the cOC/ucOC ratio.

FIG. 10: The sum of cOC and ucOC.

### DEFINITIONS

As used herein, the term “menaquinone 7” refers to compounds have the structure: 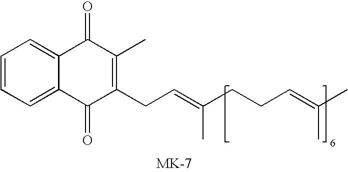
and derivatives thereof. Menaquinone 7 can also be referred to as 2-methyl-3-all-trans-farnesyl digeranyl-1,4-naphthoquinone or by its systematic name (all-E)-2-(3,7,11,15,19,23,27-Heptamethyl-2,6,10,14,18,22,26-octacosaheptaenyl)-3-methyl-1,4-naphthalenedione

As used herein, the term “natto” refers to soybean product fermented with Bacillus subtilis natto.

As used herein, the term “subject” refers all animals, including humans.

As used herein, the term “physiologically acceptable carrier” refers to any carrier or excipient commonly used with pharmaceuticals. Such carriers or excipients include, but are not limited to, oil, starch, sucrose and lactose.

As used herein, the term “oral delivery vehicle” refers to any means packaging a composition for oral administration, including, but not limited to, capsules, pills, and tablets.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

As used herein, the term “functional food” refers to a food product to which a biologically active supplement has been added.

As used herein, the term “infant food” refers to a food product formulated for an infant such as formula.

As used herein, the term “elderly food” refers to a food product formulated for persons of advanced age.

As used herein, the term “pregnancy food” refers to a food product formulated for pregnant women.

As used herein, the term “nutritional supplement” refers to a food product formulated to be used as part of a diet.

### DETAILED DESCRIPTION OF THE INVENTION

The present invention relates to the field of human nutrition, and in particular to oral delivery vehicles and nutritional supplements comprising an effective daily dose of menaquinone 7. The present inventors have found that an effective daily dose of natural menaquinone 7 provided via an oral delivery vehicle has the same bioequivalency as Natto. It is surprising that the capsules of Natto extract supported circulating levels of MK-7 that were similar to those observed with Natto food in light the suspected probiotic effect of the bacteria present in the Natto food.

The present invention is not limited to any particular mechanism of action. Indeed, an understanding of the mechanism of action is not necessary. However, the function of vitamin K is to serve as a cofactor for the enzyme gammaglutamyl carboxylase. During the vitamin K-dependent reaction, the so-called Gla-proteins are formed. Hepatic Gla-proteins are coagulation factors, Gla-proteins formed in bone and vessel wall are osteocalcin and matrix Gla-protein (MGP), respectively. Inadequate supply of vitamin K results in the occurrence of incompletely carboxylated Gla-proteins, which are biologically inactive. Various clinical studies have shown that vitamin K has an important role in the maintenance of bone and vascular health. A population-based study demonstrated an inverse correlation between vitamin K2 intake and cardiovascular mortality. Intervention studies in postmenopausal women resulted in a 40% lower rate of bone loss in vitamin K1-supplemented groups.

The present inventors have examined the effect of natto K2 capsules (rich in MK-7) on the Gla-content of the circulating bone Gla-protein osteocalcin. Osteocalcin is exclusively synthesized by the osteoblast and the active form contains three Gla-residues. The vitamin K content of the Western diet is insufficient, however, to support full carboxylation in the majority of the population. Therefore, a substantial fraction of the circulating osteocalcin is incompletely carboxylated. Under-carboxylated osteocalcin (ucOC) is a marker for sub-clinical vitamin K-deficiency of bone tissue, for low bone mineral density, and for increased fracture risk. As described above, the have demonstrated that MK-7 from natto capsules is efficiently absorbed (comparable to that from raw natto), and the absorption was much better than that of vitamin K1 from green vegetables. The present inventors have also discovered the following novel uses for menaquinone 7, especially natural menaquinone 7 compositions derived from natto: 1) use to increase and maintain vitamin K levels in serum of from about 6,000 to about 10,000 pmol/liter; 2) use to decrease the amount of uncarboxylated asteocalsin in the serum; and 3) use to decrease bone turnover.

I. Sources of Menaquinone 7

The present invention contemplates the use of menaquinone 7 from a variety of sources. In some embodiments, the menaquinone 7 of the present invention is derived from natural sources. In some preferred embodiments, the menaquinone 7 is provided as an extract from natto. Preferred extracts are available from Nattokin, Tokyo, Japan, and Natural AS, Oslo, Norway. The present invention is not limited to any particular mechanism. Indeed, an understanding of the mechanism of action is not necessary to practice the present invention. Nevertheless, it is contemplated that such extracts also contain other beneficial factors in addition to menaquinone 7, including natto kinase and Bacillus subtilis natto, which can function as a probiotic.

II. Formulation and Administration of Menaquinone 7

The menaquinone 7 dietary compositions of the present invention, including extracts of natto, may be provided in a variety of forms. In some embodiments, administration is oral. The menaquinone 7 compositions may be formulated with suitable carriers such as starch, sucrose or lactose in tablets, pills, dragees, capsules, solutions, liquids, slurries, suspensions and emulsions. In preferred embodiments, the menaquinone 7 is provided as an effective daily dose in an oral delivery vehicle. In some embodiments, administration of an effective daily dose of MK-7 via an oral delivery vehicle results in long-term circulating levels (i.e., circulating levels of MK-7 after 96 hours of daily administration of the daily effective dose) of MK-7 in the serum of between 0.1 and 50 ng/ml, preferably between about 0.2 and 25 ng/ml, and most preferably between about 0.5 and 20 ng/ml (ng MK-7 in 1 ml of serum). In other embodiments, administration of an effective daily dose of MK-7 via an oral delivery vehicle results in long-term circulating levels of MK-7 in the serum of at least 0.1 ng/ml, preferably at least 2 ng/ml, and most preferably at least about 5.0 ng/ml (ng MK-7 in 1 ml of serum). In preferred embodiments, the effective daily dose delivered via an oral delivery vehicle provides continuous circulating levels of MK-7 of in the serum of between 0.1 and 50 ng/ml, preferably between about 0.2 and 25 ng/ml, and most preferably between about 0.5 and 20 ng/ml (ng MK-7 in 1 ml of serum). In further preferred embodiments, the effective daily dose delivered via an oral delivery vehicle provides continuous circulating levels of MK-7 of in the serum of at least 0.1 ng/ml, preferably at least 2 ng/ml, and most preferably at least about 5.0 ng/ml (ng MK-7 in 1 ml of serum).

Oral delivery vehicles (e.g., tablets or capsules) of the present invention may be coated with an enteric coating which dissolves at a pH of about 6.0 to 7.0. A suitable enteric coating which dissolves in the small intestine but not in the stomach is cellulose acetate phthalate. In some embodiments, the menaquinone 7 is provided as soft gelatin capsules containing about 1 to about 1000 micrograms of menaquinone 7, preferably about 1 to 500 micrograms of menaquinone 7, more preferably about 10 to 200 micrograms menaquinone 7 and most preferably about 10 to 100 micrograms menaquinone 7. The menaquinone 7 may also be provided by any of a number of other routes, including, but not limited to, intravenous, intramuscular, intra-arterial, intramedullary, intrathecal, intraventricular, transdermal, subcutaneous, intraperitoneal, intranasal, enteral, topical, sublingual or rectal means. Further details on techniques for formulation for and administration and administration may be found in the latest edition of Remington's Pharmaceutical Sciences (Maack Publishing Co., Easton, Pa.).

Menaquinone 7, for example in an effective daily dose, may also be provided as a supplement in various food products, including animal feeds, human functional food products, infant food products, nutritional supplements, and drinks. For the purposes of this application, food products containing menaquinone 7 compositions means any natural, processed, diet or non-diet food product to which exogenous menaquinone 7 has been added. Therefore, menaquinone 7 may be directly incorporated into various prepared food products, including, but not limited to diet drinks, diet bars, supplements, prepared frozen meals, candy, snack products (e.g., chips), prepared meat products, milk, cheese, yogurt, bread, cereal and any other suitable foods.

The menaquinone 7 of the present invention may also be formulated with a number of other compounds. These compounds and substances add to the palatability or sensory perception of the particles (e.g., flavorings and colorings) or improve the nutritional value of the particles (e.g., minerals, vitamins, phytonutrients, antioxidants, etc.).

In further embodiments, the particles comprise at least one food flavoring such as acetaldehyde (ethanal), acetoin (acetyl methylcarbinol), anethole (parapropenyl anisole), benzaldehyde (benzoic aldehyde), N-butyric acid (butanoic acid), d- or l-carvone (carvol), cinnamaldehyde (cinnamic aldehyde), citral (2,6-dimethyloctadien-2,6-al-8, gera-nial, neral), decanal (N-decylaldehyde, capraldehyde, capric aldehyde, caprinaldehyde, aldehyde C-10), ethyl acetate, ethyl butyrate, 3-methyl-3-phenyl glycidic acid ethyl ester (ethyl-methyl-phenyl-glycidate, strawberry aldehyde, C-16 aldehyde), ethyl vanillin, geraniol (3,7-dimethyl-2,6 and 3,6-octadien-1-ol), geranyl acetate (geraniol acetate), limonene (d-, l-, and dl-), linalool (linalol, 3,7-dimethyl-1,6-octadien-3-ol), linalyl acetate (bergamol), methyl anthranilate (methyl-2-aminobenzoate), piperonal (3,4-methylenedioxy-benzaldehyde, heliotropin), vanillin, alfalfa (Medicago sativa L.), allspice (Pimenta officinalis), ambrette seed (Hibiscus abelmoschus), angelic (Angelica archangelica), Angostura (Galipea officinalis), anise (Pimpinella anisum), star anise (Illicium verum), balm (Melissa officinalis), basil (Ocimum basilicum), bay (Laurus nobilis), calendula (Calendula officinalis), (Anthemis nobilis), capsicum (Capsicum frutescens), caraway (Carum carvi), cardamom (Elettaria cardamomum), cassia, (Cinnamomum cassia), cayenne pepper (Capsicum frutescens), Celery seed (Apium graveolens), chervil (Anthriscus cerefolium), chives (Allium schoenoprasum), coriander (Coriandrum sativum), cumin (Cuminum cyminum), elder flowers (Sambucus canadensis), fennel (Foeniculum vulgare), fenugreek (Trigonella foenum-graecum), ginger (Zingiber officinale), horehound (Marrubium vulgare), horseradish (Armoracia lapathifolia), hyssop (Hyssopus officinalis), lavender (Lavandula officinalis), mace (Myristica fragrans), marjoram (Majorana hortensis), mustard (Brassica nigra, Brassica juncea, Brassica hirta), nutmeg (Myristica fragrans), paprika (Capsicum annuum), black pepper (Piper nigrum), peppermint (Mentha piperita), poppy seed (Papayer somniferum), rosemary (Rosmarinus officinalis), saffron (Crocus sativus), sage (Salvia officinalis), savory (Satureia hortensis, Satureia montana), sesame (Sesamum indicum), spearmint (Mentha spicata), tarragon (Artemisia dracunculus), thyme (Thymus vulgaris, Thymus serpyllum), turmeric (Curcuma longa), vanilla (Vanilla planifolia), zedoary (Curcuma zedoaria), sucrose, glucose, saccharin, sorbitol, mannitol, aspartame. Other suitable flavoring are disclosed in such references as Remington's Pharmaceutical Sciences, 18th Edition, Mack Publishing, p. 1288-1300 [1990], and Furia and Pellanca, Fenaroli's Handbook of Flavor Ingredients, The Chemical Rubber Company, Cleveland, Ohio, [1971], known to those skilled in the art.

In other embodiments, the particles comprise at least one synthetic or natural food coloring (e.g., annatto extract, astaxanthin, beet powder, ultramarine blue, canthaxanthin, caramel, carotenal, beta carotene, carmine, toasted cottonseed flour, ferrous gluconate, ferrous lactate, grape color extract, grape skin extract, iron oxide, fruit juice, vegetable juice, dried algae meal, tagetes meal, carrot oil, corn endosperm oil, paprika, paprika oleoresin, riboflavin, saffron, tumeric, tumeric and oleoresin).

In still further embodiments, the particles comprise at least one phytonutrient (e.g., soy isoflavonoids, oligomeric proanthcyanidins, indol-3-carbinol, sulforaphone, fibrous ligands, plant phytosterols, ferulic acid, anthocyanocides, triterpenes, omega 3/6 fatty acids, conjugated fatty acids such as conjugated linoleic acid and conjugated linolenic acid, polyacetylene, quinones, terpenes, cathechins, gallates, and quercitin). Sources of plant phytonutrients include, but are not limited to, soy lecithin, soy isoflavones, brown rice germ, royal jelly, bee propolis, acerola berry juice powder, Japanese green tea, grape seed extract, grape skin extract, carrot juice, bilberry, flaxseed meal, bee pollen, ginkgo biloba, primrose (evening primrose oil), red clover, burdock root, dandelion, parsley, rose hips, milk thistle, ginger, Siberian ginseng, rosemary, curcumin, garlic, lycopene, grapefruit seed extract, spinach, and broccoli.

In still other embodiments, the particles comprise at least one vitamin (e.g., vitamin A, thiamin (B1), riboflavin (B2), pyridoxine (B6), cyanocobalamin (B12), biotin, ascorbic acid (vitamin C), retinoic acid (vitamin D), vitamin E, folic acid and other folates, vitamin K, niacin, and pantothenic acid). In some embodiments, the particles comprise at least one mineral (e.g., sodium, potassium, magnesium, calcium, phosphorus, chlorine, iron, zinc, manganese, flourine, copper, molybdenum, chromium, selenium, and iodine). In some particularly preferred embodiments, a dosage of a plurality of particles includes vitamins or minerals in the range of the recommended daily allowance (RDA) as specified by the United States Department of Agriculture. In still other embodiments, the particles comprise an amino acid supplement formula in which at least one amino acid is included (e.g., l-carnitine or tryptophan).

In further embodiments of the present invention, the menaquinone 7 of the present invention is provided as a powdered extract of natto. The extract is formed into a powder by methods such as freeze drying or spray drying (See, e.g., U.S. Pat. No. 4,232,052, incorporated herein by reference). In general, spray drying involves liquefying or emulsifying a substance and then atomizing it so that all but a small percentage of water is removed, yielding a free flowing powder. Suitable spray drying units include both high pressure nozzle spray driers and spinning disk or centrifugal spray driers. The present invention is not limited to any particular excipient. Indeed, a variety of excipients are contemplated, including, but not limited to, starch, lactose, HI-CAP 100 (National Starch, Bridgewater, N.J.) and HI-CAP 200 (National Starch, Bridgewater, N.J.). In some preferred embodiments, the natto extract is handled away from daylight. In still firther embodiments, the natto extracts are microencapsulated by methods known in the art.

III. Uses of Compositions Comprising Menaquinone 7

The present invention contemplates the use of an effective daily dose of menaquinone 7 to treat a variety of disorders. In some embodiments, the effective daily dose of menaquinone 7 is provided to a patient (e.g., an animal, including humans). In preferred embodiments, the effective daily dose is administered orally. In some embodiments, the menaquinone 7 is used to reduce bone pain in a patient. The effective daily dose of menaquinone 7 of the present invention may also be used for indications such as reduction of the effects of osteoporosis, increase of bone density, prevention of arterial calcification, prevention of high blood pressure, prevention of strokes, prevention of heart attacks, prevention of senility, and prevention of cancer. Additionally, extracts of natto have a probiotic effect due to the presence of Bacillus subtilis natto. As such, the natto extracts confer benefits such as antibiotic activity towards such pathogens as typhoid bacilli, amoebic dysentery, and Escherichia coli 0-157, and also improves digestion and prevents intestinal disorders. Accordingly, the present invention provides methods that comprise providing a patient suffering from or at risk of one of the foregoing conditions and an effective daily dose of menaquinone 7 and administering the daily dose of menaquinone 7 to the patient under conditions such that the condition is relieved. In some embodiments, the menaquinone 7 is used prophylactically to prevent one or more of the foregoing conditions.

The present inventors have found that administration of menaquinone 7 results in a more stable plasma vitamin K level, which is substantially higher than K1 concentrations after a similar dose of K1. It is known that after intestinal absorption, K1 transported in the blood stream via the triglycerides, which are cleared by the liver within a few hours. Shortly after intake, also K2-vitamins are found in the triglycerides, but—unlike K1—they are re-distributed into the circulation via the low density lipoproteins. Without being limited to any mechanism, it is believed that the circulating MK-7 levels remained high for longer periods. As a result, extra-hepatic cells benefit more from menaquinone 7 than from K1. Accordingly, the present invention provides methods for increasing serum levels of vitamin K species such as menaquinone 7 to greater than 6,000 pmol/liter, preferably between about 6,000 pmol/liter and about 10,000 pmol/liter, by administering the menaquinone 7 compositions described above.

The present inventors have also discovered that administration of the menaquinone 7 compositions of the present invention to subjects decreases the amount of under-carboxylated osteocalcin (ucOC) in serum. Without being limited to any particular mechanism or theory, high levels of under-carboxylated osteocalcin have been demonstrated by many independent groups to be a strong marker for low bone mass and increased hip fracture risk. The ratio between carboxylated and under-carboxylated osteocalcin is a generally accepted marker for bone vitamin K status. The present inventors have discovered that both K1 and menoquinone 7 induced a comparable improvement of this ratio, although the shape of the curves was different. For K1 the effect was quick and had reached plateau levels after 3 days. For menaquinone 7, on the other hand, the ratio increased gradually with mean values crossing the K1 curve at week 5. There was no indication for a levelling off of the curve, so it is expect that the improvement may continue after longer periods of menaquinone 7 intake. Thus, the maximal effects of menaquinone 7 supplementation are only obtained if the supplement is taken on a regular basis for longer periods of time (probably life-long). Accordingly, in some embodiments, the present invention provides methods for decreasing the amount of under-carboxylated osteocalcin in serum by supplementing the diet with menaquinone 7 compositions.

In sill other embodiments, the present invention provides methods for decreasing bone turnover by the administration of menaquinone 7. A most remarkable and unexpected difference between K1 and menaquinone 7 was that only the latter formulation seemed to induce a decrease in bone turnover. Since high bone turnover is generally agreed as one of the main factors for high bone loss and development of osteoporosis, all clinical treatments of postmenopausal osteoporosis are aimed at decreasing bone turnover. It must be mentioned that the marker used (cOC+ucOC) is not an ideal marker to reach our conclusion, and ideally one would need to monitor additional markers such as total osteocalcin (direct measurement of all species including partially carboxylated ones), bone-specific alkaline phosphatase, as well as N-terminal and C-terminal collagen degradation products (NTX and CTX, respectively).

### EXPERIMENTAL

### Example 1

This example describes the circulating levels of menaquinone 7 (MK-7) in humans consuming either natto or capsules containing extracts of natto. An open study among 6 volunteers in a cross-over design (2×3) was performed. On Monday morning participants received a single dose of either natto food (from the local oriental shop) or natto capsules. The natto food contained 1181 μg of MK-7, the natto capsules contained 1130 μg of MK-7. Blood samples were taken at t=0, 2, 4, 6, 8, 24, 48, 72, and 96 h. All samples were analysed for MK-7.

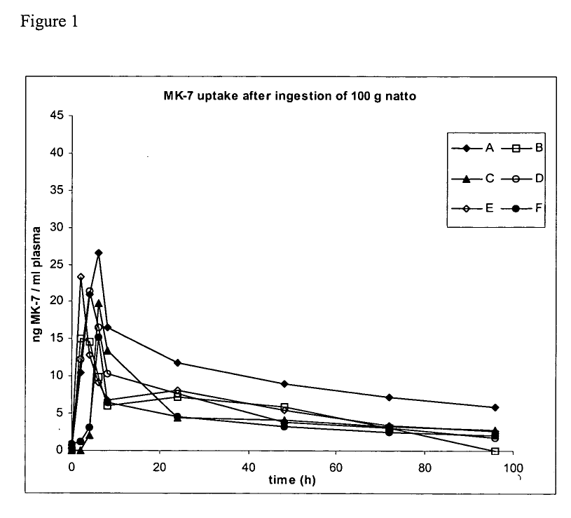

The circulating MK-7 concentrations in all 6 volunteers after eating 100 g of natto food are given in Table 1 and FIG. 1. All concentrations are in ng/mL.

TABLE 1

| time (h) | A | B | C | D | E | F | average |
|---|---|---|---|---|---|---|---|
| 0 | 0.3 | 0.0 | 0.0 | 0.5 | 0.7 | 0.8 | 0.4 |
| 2 | 10.5 | 15.1 | 0.0 | 12.2 | 23.3 | 1.1 | 10.4 |
| 4 | 20.9 | 14.6 | 2.1 | 21.3 | 12.9 | 3.0 | 12.5 |
| 6 | 26.6 | 10.0 | 19.8 | 16.6 | 9.1 | 15.2 | 16.2 |
| 8 | 16.5 | 6.1 | 13.4 | 10.3 | 6.8 | 6.5 | 9.9 |
| 24 | 11.8 | 7.3 | 4.5 | 7.7 | 8.0 | 4.5 | 7.3 |
| 48 | 9.0 | 5.9 | 4.1 | 3.8 | 5.4 | 3.2 | 5.2 |
| 72 | 7.3 | 3.1 | 3.3 | 3.1 | 3.4 | 2.5 | 3.8 |
| 96 | 5.9 | 0.0 | 2.7 | 1.7 | 2.6 | 2.1 | 2.5 |

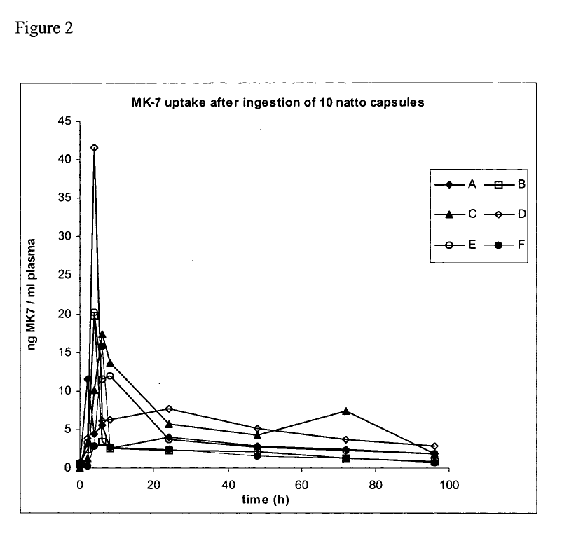

The circulating MK-7 concentrations in all 6 volunteers after eating 10 natto capsules are given in Table 2 and FIG. 2. All concentrations are in ng/mL

TABLE 2

| time (h) | A | B | C | D | E | F | average |
|---|---|---|---|---|---|---|---|
| 0 | 0.7 | 0.6 | 0 | 0.5 | 0.3 | 0.5 | 0.4 |
| 2 | 11.5 | 2.4 | 1.3 | 3.8 | 3.2 | 0.3 | 3.8 |
| 4 | 4.4 | 19.8 | 10.1 | 41.5 | 20.3 | 2.9 | 16.5 |
| 6 | 5.6 | 3.4 | 17.4 | 6.1 | 11.5 | 15.8 | 10 |
| 8 | 2.6 | 2.6 | 13.7 | 6.3 | 12 | 2.8 | 6.7 |
| 24 | 4 | 2.3 | 5.7 | 7.7 | 3.7 | 2.4 | 4.3 |
| 48 | 2.8 | 2.2 | 4.3 | 5.2 | 2.7 | 1.6 | 3.1 |
| 72 | 2.4 | 1.3 | 7.4 | 3.7 | 2.3 | 1.2 | 3 |
| 96 | 1.8 | 0.8 | 1.8 | 2.9 | 1.8 | 0.8 | 1.7 |

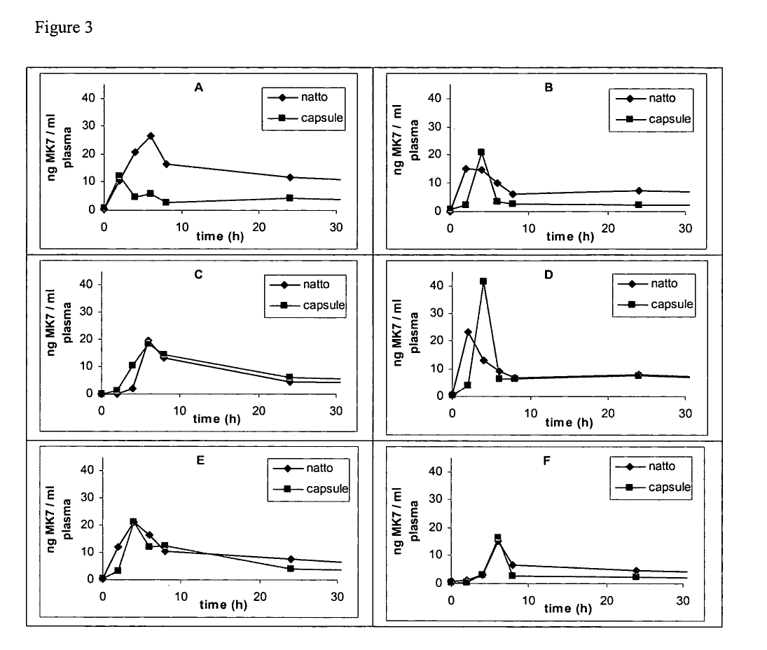

In FIG. 3 the individual values (first day only) are provided for natto food and natto capsules per subject. It is immediately clear that subject A has a very different uptake pattern for natto food and natto capsules. This may be related to the subject's life style life style which includes intense physical activity which may have affected the subject's metabolism on some days. For all calculations we have included these data. The effect on the means is negligible, and the conclusions remain the same whether or not these data are included. Furthermore, all data for natto capsules has been multiplied by 1181/1130, which compensates for the different MK-7 intake in the natto and capsule regimen.

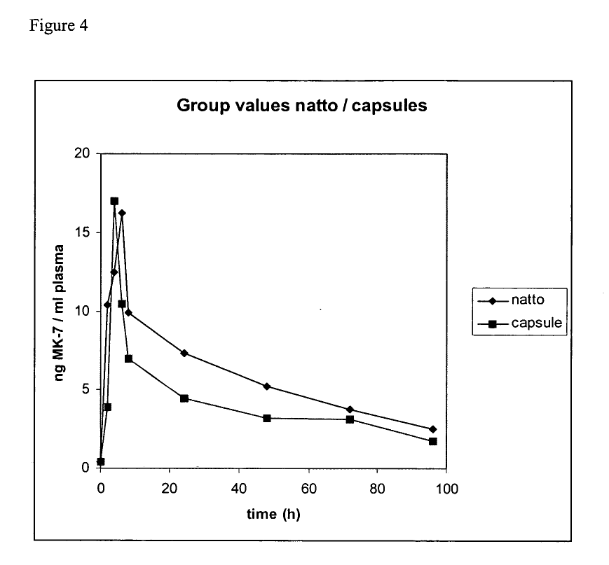

In FIG. 4 the mean values for the group are plotted. This curve shows that there is no real difference between natto food and natto capsules with respect to their delivery of MK-7 into the blood stream. The number of participants is too low to evaluate whether slight differences are statistically significant. If there are differences at all, these differences are not believed to be nutritionally relevant at this time.

Another way to analyze these data is to determine the Area Under the Curve (AUC). This has been done for two periods: period 0-8 h (first absorption phase) and the period 24-96 h (long time effects). The results are shown in Table 3.

TABLE 3

| | AUC 0-8 | AUC 0-8 | AUC 24-96 | AUC 24-96 |
|---|---|---|---|---|
| Subject | natto food | capsules | natto food | capsules |
| A | 132.8 | 48.6 | 603.6 | 202.8 |
| B | 85.5 | 57.1 | 303.6 | 127.2 |
| C | 57.2 | 75.0 | 264.0 | 387.6 |
| D | 98.1 | 109.6 | 338.4 | 340.8 |
| E | 111.1 | 86.4 | 278.4 | 194.4 |
| F | 52.4 | 41.0 | 216.0 | 111.6 |
| Average | 89.5 | 69.6 | 334.0 | 227.4 |
| SD | 28.4 | 23.5 | 126.2 | 103.0 |

For the AUC 0-8 the absorption from capsules was 78% that of natto, for the AUC 24-96 it was 68%. Although the differences are not statistically significant (Wilcoxon test for paired observations), there is a tendency for better absorption from natto food. We think that this may be related with the higher bulk of material ingested from natto food. As a result, bile secretion may be stimulated more. Maybe the structure of the food matrix plays also a role. From the data we may rule out the possibility that natto food has a higher probiotic effect than natto capsules: the AUC 24-96 is 3.7 fold that of the AUC 0-8 in the case of natto food, and 3.3 fold in the case of capsules.

An impressive advantage of both natto food and natto capsules over vitamin K1 or MK-4 is that the latter vitamins are rapidly taken up from the circulation (probably by the liver), so that after 8 h postprandially the remaining circulating concentrations are really low. Unlike K1 and MK-4, MK-7 is incorporated into low density lipoproteins (LDL) where it remains available for extrahepatic uptake during periods of several days. It is surprising that the capsules of Natto extract supported circulating levels of MK-7 that were similar to those observed with Natto food in light the suspected probiotic effect of the bacteria present in the Natto food. This probiotic effect has been documented by Kaneki et al., Nutrition 17:315-321 (2001).

### Example 2

An open study was conducted in which 12 volunteers were randomized in two groups receiving either 180 μg MK-7 (menaquinone 7, 0.27 μmol) or 150 μg K1 (0.33 μmol) per day. The MK-7 was obtained from 2 nattoK2 capsules (Natural ASA) per day, the K1 from a pharmacological preparation (Roche). The supplements were taken at 18.00 h of each day during the 6-week study period. Blood was taken between 08.00 and 09.00 h of days 0 (baseline), 3, 7, 14, 21, 28, 35, and 42. A non-treated control group (n=3) served as a reference group, with blood drawings on days 0, 14, 28, and 42.

Measurements: vitamin K1, MK-7, under-carboxylated osteocalcin (ucOC) and carboxylated osteocalcin (cOC).

Study population: 6 men and 6 women, aged 55-60 (women postmenopausal). Non-treated controls: 1 man, 2 women in the same age range.

Results:

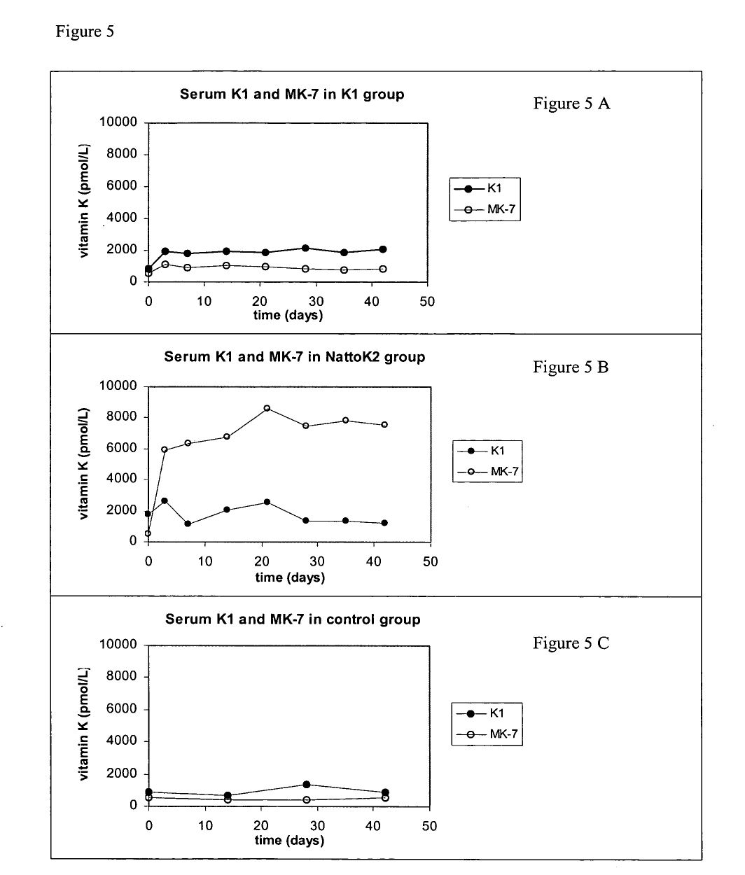

MK-7 and K1 have different molecular weights (650 and 450, respectively). Therefore, all doses and concentrations were calculated on a molar basis. Since the daily dose of MK-7 from natto was 18% lower than that from K1 (0.27 and 0.33 μmol/day, respectively), it should be kept in mind that the expected results for natto capsules are similarly lower compared to those for K1. FIG. 5 shows the mean serum vitamin K concentration (absolute values) in the three groups: the K1-treated group (5A), the nattoK2-treated group (5B) and the controls (5C). The normal ranges for K1 and MK-7 in non-treated subjects are between 500 and 1000 pmol/L (FIG. 5C). In the K1 group the serum K1 levels had increased from 816 to around 2000 pmol/L, and the plateau value was reached within the first three days. The short half-lime time of K1 is the reason for high serum K1 levels shortly after intake, followed by near-baseline levels at 15 h after ingestion. In the nattoK2 MK-7 preparation group, relatively high levels of circulating MK-7 were found, with an increase from 538 pmol/L at baseline to 6000 pmol/L at day 3, before reaching a plateau level of around 7000 pmol/L after 2 weeks. This is consistent with the much longer half-life time of MK-7 in the circulation, and demonstrates that with nattoK2 higher plasma vitamin K levels are reached, which are stable during the day. Hence vitamin K from the nattoK2 MK-7 preparation is more available for bone and vascular cells than is K1. Due to the limited number of participants, the mean baseline levels in the various groups may differ. Therefore, only treatment-induced changes will be presented in the following figures.

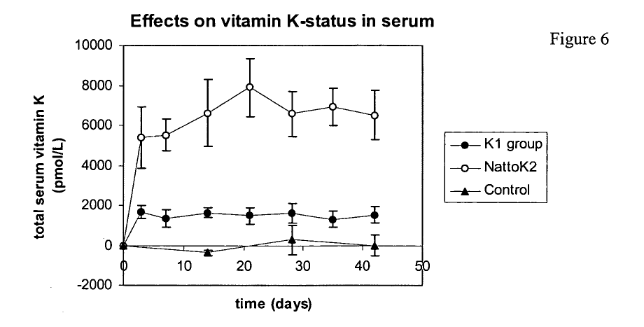

The effects of pharmacological K1 and nattoK2 MK-7 preparation on the total vitamin K status of the subjects is given in FIG. 6. All data are given as change relative to baseline. Whereas in the controls the vitamin K status remained constant (around 1450 pmol/L) during the 6 weeks of the experiment, pharmacological K1 supplements induced an increase of 1500 pmol/L (mean plateau value). Although the total vitamin K dose in the NattoK2 group was 18% lower, this product induced a markedly higher increase in total vitamin K-status, with a plateau level up to 6900 pmol/L above the baseline values. This is about 7 fold better than K1.

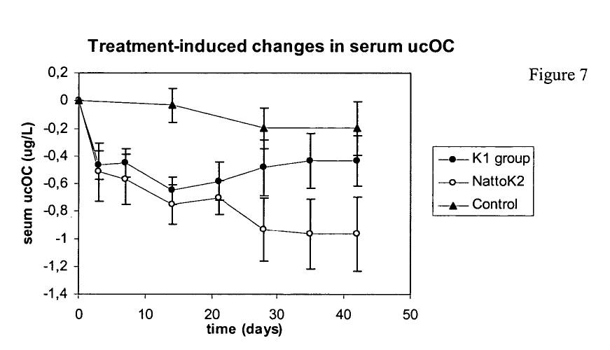

FIG. 7 shows the treatment-induced changes in serum undercarboxylated osteocalcin (ucOC). In the controls there was a minor decrease, but this was not different from the baseline. K1 treatment induced a decrease of ucOC from 2.1 to 1.7 μg/L, the latter value being reached within three days. NattoK2 treatment resulted in a more pronounced decrease of ucOC from 1.9 μg/L at baseline to values below 1.0 μg/L during the last three weeks of treatment. Both K1 and NattoK2 induce a decrease of serum ucOC. Since serum ucOC was demonstrated to be strongly associated with hip fracture risk, these data suggest that increased vitamin K intake improves bone health. Although the dose of MK-7 in NattoK2 was lower than that of K1, the effect of Natto K2 was more pronounced (about two-fold) compared to that of K1.

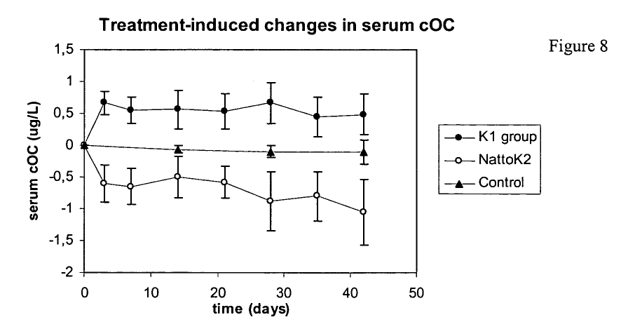

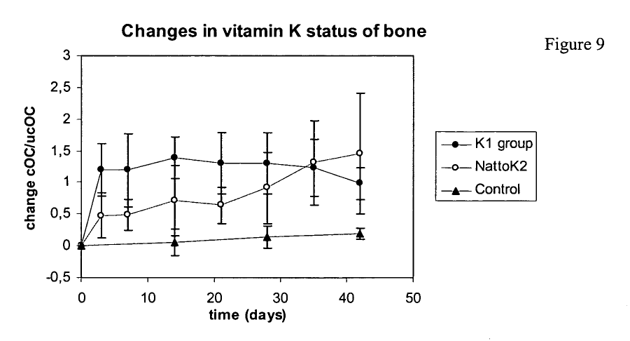

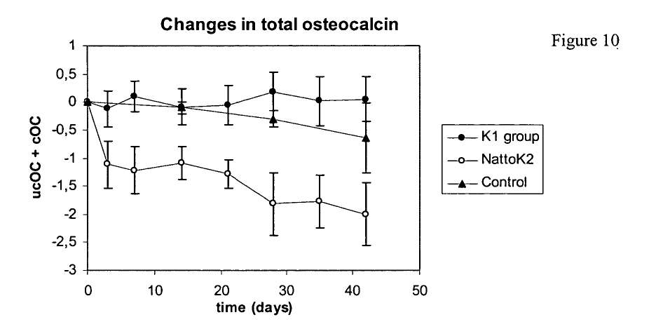

FIG. 8 shows the treatment-induced changes in serum carboxylated osteocalcin. These values are needed to calculate the vitamin K-status in bone (the ratio cOC/ucOC) and to estimate the bone metabolic activity (cOC+ucOC). Here a remarkable difference between the effects of K1 and nattoK2 (i.e.: MK-7) was found: whereas cOC was increased by K1 supplementation, it was decreased by nattoK2. The importance of this finding will become clear from FIGS. 9 and 10.

FIG. 9 shows the treatment-induced changes in the vitamin K status of bone, expressed as the cOC/ucOC ratio. The effect of K1 seemed to be at its maximum quicker than that of MK-7 from nattoK2 MK-7 preparation, which showed a more gradual increase. After 6 weeks the effect of NattoK2 was somewhat larger than that of K1 and, more importantly, it seemed to be rising still. This suggests that a maximal effect of NattoK2 MK-7 preparation is only obtained if it is taken on a regular basis. Accordingly, vitamin K1 and NattoK2 MK-7 preparation have similar effects on the vitamin K status of bone.

FIG. 10 shows the treatment of the sum of cOC and ucOC. Although it is doubtful whether this sum may be used as a real marker for total osteocalcin, it is probably a good estimate for total bone metabolism. Surprisingly, there was a large difference between the effects of K1 and nattoK2 MK-7 preparation on bone turnover measured as ucOC+cOC: whereas bone turnover remained constant during the vitamin K1 regimen (from 5.28 to 5.33), it strongly decreased (from 5.45 to 3.45) in the nattoK2 MK-7 preparation group. Also in the control group bone turnover remained constant. A decrease in bone turnover is generally regarded as protective against further bone loss and osteoporosis. Accordingly, it appears that nattoK2 MK-7 preparation, and not K1 decreases bone turnover. This is an unexpected effect that may explain a higher value of nattoK2 MK-7 preparation for bone health than formulations containing K1.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in medicine, biochemistry, or related fields are intended to be within the scope of the following claims.

1. An oral delivery vehicle comprising an effective daily dose of menaquinone 7, wherein said effective daily dose is from about 10 to about 200 micrograms of menaquinone 7.

2. The oral delivery vehicle of claim 1, wherein said menaquinone 7 is derived from natto.

3. The oral delivery vehicle of claim 1, further comprising natto kinase.

4. The oral delivery vehicle of claim 1 provided as a tablet.

5. The oral delivery vehicle of claim 1, provided as a gel capsule.

6. The oral delivery vehicle of claim 1, comprising an enteric coating.

7. A food product comprising added menaquinone 7.

8. The food product of claim 7, comprising an effective daily dose of menaquinone 7, wherein said effective daily dose is from about 10 to about 200 micrograms of menaquinone 7.

9. The food product of claim 7, wherein said menaquinone 7 is provided as a natto extract.

10. A nutritional supplement comprising an effective daily dose of menaquinone 7, wherein said effective daily dose is from about 10 to about 200 micrograms of menaquinone 7.

11. The nutritional supplement of claim 10, wherein said menaquinone 7 is provided as a natto extract.

12. A method comprising:
a) providing
i) a subject, and
ii) the oral delivery vehicle of claim 1,
b) administering said pharmaceutical composition to said subject.

13. The method of claim 12, wherein said administering is oral.

14. The method of claim 12, wherein said menaquinone 7 in said oral delivery vehicle is derived from natto.

15. The method of claim 12, wherein said oral delivery is a tablet.

16. The method of claim 12, wherein said oral delivery vehicle is a gel capsule.

17. The method of claim 12, wherein said oral delivery vehicle comprises an enteric coating.

18. The method of claim 12, wherein said oral delivery vehicle further comprises natto kinase.

19. A method of relieving bone pain comprising:
a) providing
i) a patient, and
ii) a composition comprising an effective amount of menaquinone 7,
b) administering said composition to said patient under conditions such that said bone pain is reduced.

20. The method of claim 19, wherein said administering is oral.

21. The method of claim 19, wherein said effective dose of menaquinone 7 is about 10 to about 200 micrograms.

22. The method of claim 19, wherein said menaquinone 7 in said composition is derived from natto.

23. The method of claim 19, wherein said composition is provided as a tablet.

24. The method of claim 19, wherein said composition is provided as a gel capsule.

25. The method of claim 19, wherein said composition comprises an enteric coating.

26. The method of claim 19, wherein said composition further comprises natto kinase.

27. An oral delivery vehicle comprising an effective daily dose of menaquinone 7, wherein said effective daily dose supports a circulating concentration of menaquinone-7 in the body of about 0.1 to 50 ng menaquinone-7 per milliliter of serum.

28. The oral delivery vehicle of claim 27, wherein said menaquinone 7 is derived from natto.

29. The oral delivery vehicle of claim 27, further comprising natto kinase.

30. The oral delivery vehicle of claim 27, wherein said oral delivery vehicle is a tablet.

31. The oral delivery vehicle of claim 27, wherein said oral delivery vehicle is a gel capsule.

32. The oral delivery vehicle of claim 27, comprising an enteric coating.

33. A method of decreasing under-carboxylated osteocalcin in a subject comprising:
a) providing
i) a subject, and
ii) a composition comprising an effective amount of menaquinone 7,
b) administering said composition to said subject under conditions such that said under-carboxylated osteocalcin is reduced.

34. The method of claim 33, wherein said administering is oral.

35. The method of claim 33, wherein said effective dose of menaquinone 7 is about 10 to about 200 micrograms.

36. The method of claim 33, wherein said menaquinone 7 in said composition is derived from natto.

37. The method of claim 33, wherein said composition is provided as a tablet.

38. The method of claim 33, wherein said composition is provided as a gel capsule.

39. The method of claim 33, wherein said composition comprises an enteric coating.

40. The method of claim 33, wherein said composition further comprises natto kinase.

41. A method of decreasing bone turnover in a subject comprising:
a) providing
i) a subject, and
ii) a composition comprising an effective amount of menaquinone 7,
b) administering said composition to said subject under conditions such that said bone turnover is reduced.

42. The method of claim 41, wherein said administering is oral.

43. The method of claim 41, wherein said effective dose of menaquinone 7 is about 10 to about 200 micrograms.

44. The method of claim 41, wherein said menaquinone 7 in said composition is derived from natto.

45. The method of claim 41, wherein said composition is provided as a tablet.

46. The method of claim 41, wherein said composition is provided as a gel capsule.

47. The method of claim 41, wherein said composition comprises an enteric coating.

48. The method of claim 41, wherein said composition further comprises natto kinase.

49. The method of claim 41, wherein said decrease in bone turnover is indicated by an increase in total osteocalcin determined as the sum of carboxylated and uncarboxylated osteocalcin.

50. The method of claim 41, wherein said increase in total osteocalcin is determined by additionally summing partially carboxylated osteocalcin with said sum of carboxylated and uncarboxylated osteocalcin.

51. A method of increasing serum menaquinone 7 levels to greater than 6000 pmol/L in a subject comprising:
a) providing
i) a subject, and
ii) the oral delivery vehicle of claim 1,
b) administering said oral delivery vehicle to said subject under conditions such that serum menaquinone levels are increased to about 6000 pmol/L.

52. The method of claim 51, wherein said administering is oral.

53. The method of claim 51, wherein said menaquinone 7 in said oral delivery vehicle is derived from natto.

54. The method of claim 51, wherein said oral delivery is a tablet.

55. The method of claim 51, wherein said oral delivery vehicle is a gel capsule.

56. The method of claim 51, wherein said oral delivery vehicle comprises an enteric coating.

57. The method of claim 51, wherein said oral delivery vehicle further comprises natto kinase.

58. The method of claim 51, further comprising maintaining said serum menaquinone 7 levels at 6000 pmol/liter or greater.

59. The method of claim 51, wherein said menaquinone levels are increased to about 6,000 to about 10,000 pmol/liter.

60. The method of claim 58, wherein said menaquinone levels are maintained at about 6,000 to about 10,000 pmol/liter.
